(19) United States
(12) Patent Application Publication
(10) Pub. No.: US 2024/0287542 A1
(43) Pub. Date: Aug. 29, 2024

(54) AAV VECTOR VARIANTS FOR OCULAR GENE DELIVERY

(71) Applicants: UNIVERSITÄT BERN, Bern (CH); MEDIZINISCHE HOCHSCHULE HANNOVER, Hannover (DE)

(72) Inventors: Sonja KLEINLOGEL, Bern (CH); Anand DAVID, Mumbai (IN); Hildegard BÜNING, Hannover (DE)

(21) Appl. No.: 17/755,434

(22) PCT Filed: Nov. 2, 2020
(86) PCT No.: PCT/EP2020/080704

§ 371 Date: Apr. 28, 2022

(30) Foreign Application Priority Data
Oct. 31, 2019 (EP) 19206603.3

(51) Int. Cl. C12N 15/86; A61K 9/00; A61K 38/00; A61K 48/00; A61P 27/02; C07K 14/005

(52) U.S. Cl. CPC C12N 15/86; A61K 9/0019; A61K 9/0048; A61K 48/0075; A61P 27/02; C07K 14/005; A61K 38/00; C07K 2319/33; C12N 2750/14122; C12N 2750/14143; C12N 2750/14145; C12N 2810/40

(57) ABSTRACT

The present invention relates to adeno-associated virus capsid polypeptide sequences and their use in therapeutic transgene delivery to the eye and potentially other tissues.

### RELATED APPLICATIONS

This application is a U.S. national stage entry under 35 U.S.C. § 371 of international application no. PCT/EP2020/080704, filed Nov. 2, 2020, which designates the U.S. and claims the benefit of priority of European application n. 19206603.3 filed Oct. 31, 2019, incorporated herein by reference in its entirety.

The present invention relates to adeno-associated virus capsid polypeptide sequences and their use in therapeutic transgene delivery to the eye targeting the photoreceptors or retinal pigment epithelial cells.

### SEQUENCE LISTING

The instant application contains a Sequence Listing which has been submitted electronically in ASCII format and is hereby incorporated by reference in its entirety. Said ASCII copy, created on Apr. 28, 2022, is named eolf-othd-000002 and is 65,212 bytes in size.

### BACKGROUND OF THE INVENTION

Permanent degeneration of light-sensing retinal photoreceptor cells (PRs) causes blindness. Photoreceptor death can be induced by inherited mutations localized mainly in PRs or retinal pigment epithelial (RPE) cells, such as in retinitis pigmentosa (RP), or by a multifactorial condition affecting the health of photoreceptors, such as for example in age-related macular degeneration (AMD). The genetic mutations responsible for many forms of photoreceptor degeneration are identified, enabling interventions by administration of therapeutic transgenes. Existing PR targeting nucleic acid-based therapies are effective only at the early disease stages when damage to PR is minimal. Gene therapy employing a suitable optogenetic protein that confers light sensitivity to functional inner retinal cells might potentially restore vision even in later disease stages. To make such approaches applicable in a clinical setting, improved vectors with better retinal penetration for gene delivery are required.

Recombinant vectors based on adeno-associated virus (AAV) are candidates for therapeutic gene transfer in the eye. Currently, the AAV vectors are typically applied subretinally. Subretinal application, however, has been shown to lead to a decrease in retinal thickness and visual acuity. In addition, subretinal application only achieves gene transfer and expression in cells immediately adjacent to the bulk of injected fluid, while an effective administration method should reach cells along the entire width of the retina. Intravitreal injection into the jelly-like filling of the eye (the vitreous humor), does not only have the potential to deliver the vector to the entire retina, but is also much safer and far less technically demanding than subretinal injection. However, the inner limiting membrane (ILM) that separates the neural retina from the vitreous humor is abundant in native receptors of many AAV serotypes and therefore presents a formidable barrier for AAV vectors. Engineered AAV vectors that penetrate better through the ILM and other retinal barriers when delivered intravitreally are needed to increase efficacy and safety of ocular AAV gene therapy.

Based on the above-mentioned state of the art, the objective of the present invention is to provide means and methods for gene transfer into the retina enabling targeting of all retinal cell types, particularly inner retinal cells and photoreceptors. This objective is attained by the subject-matter of the independent claims of the present specification.

### SUMMARY OF THE INVENTION

A first aspect of the invention relates to an adeno-associated virus (AAV) capsid polypeptide comprising a peptide insertion at position 453 or 587/588 of the AAV serotype 2 capsid or a position homologous thereto in an AAV capsid of another serotype (Table 3: #1, #2), located at the highest and second highest capsid protrusions, respectively. The peptide insert is selected from the following sequences:

(Cap3; SEQ ID NO 10)
SASEAST,
(Cap5; SEQ ID NO 11)
DTRPHDQ,
(Cap7; SEQ ID NO 12)
EHYNSTC,
(Cap9; SEQ ID NO 13)
PNPNCTL,
(Cap11; SEQ ID NO 14)
TPPSITA,
(Cap12; SEQ ID NO 15)
CGESSYL,
(Cap13; SEQ ID NO 16)
PRTPHTA
and
(Cap14; SEQ ID NO 17)
ELCDGFA.

The peptide may be flanked by short stretches of other amino acids (1, 2, 3, 4, 5 or even 6 AA). Particular examples of flanking amino acids to embed the indicated sequence at the indicated position within the capsid sequence can be selected from (but are not limited to) Ala, Leu, Gly, Ser, and Thr.

This peptide insert increases the infection efficacy of an adeno-associated virus and/or transduction efficacy of an adeno-associated vector displaying the peptide insert on the capsid surface at 1-587 above indicated position at least for the tested cell types and tissues.

It might as well work at 1-588 or 1-453 due to their cell surface exposure. Without wanting to be bound by theory, the inventors believe that at least part of the effect is due to lower binding to heparan sulphate proteoglycan in vivo.

A second aspect of the invention relates to a nucleic acid sequence encoding the AAV capsid polypeptide according to the first aspect. Particular embodiments include the inclusion of this nucleic acid sequence in an AAV cap sequence, particularly in an AAV serotype 2 capsid sequence for generation of a capsid-engineered AAV vector.

A third aspect of the invention relates to an agent selected from the AAV capsid polypeptide, an AAV vector, and the nucleic acid sequence for treatment of a condition affecting the retina or an RPE cell. Alternative forms of this aspect are embodied by methods of treatment of conditions affecting photoreceptors or RPE cells, comprising the administration of the agent according to the invention to a patient in need thereof.

Administration forms comprising the agents of the invention are further aspects of the invention.

### BRIEF DESCRIPTION OF THE FIGURES

FIG. 1 Retinal layer specificity of expression for selected capsid variants.

FIG. 2 Examples of pan-retinal transgene expression throughout the outer murine retina after intravitreal injection.

FIG. 3 Comparison of transgene expression delivered by novel capsids with AAV2(M6) and AAV2(7m8) after intravitreal injection into the mouse eye.

### DETAILED DESCRIPTION OF THE INVENTION

### Terms and Definitions

The abbreviation AAV in the context of the present specification relates to adeno-associated virus.

The term AAV vector in the context of the present specification relate to a viral vector composed of 60 AAV capsid proteins and an encapsidated AAV nucleic acid. An AAV vector is derived from an AAV virion, but the AAV vector is engineered to be replication-incompetent in the presence of a helper virus by removing the rep and cap genes from the AAV genome. The encapsidated AAV nucleic acid may comprise a transgene which is to be delivered into a target cell.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.) and chemical methods.

The term AAV capsid in the context of the present specification relates to a polypeptide encoded by engineered capsid (cap) genes generated by the inventors and listed herein below. The AAV capsid disclosed herein may be used to assemble recombinant adeno-associated viral vectors for gene therapy.

Reference to an amino acid position in the AAV capsid in the context of the present specification relates to the capsid amino acid sequence of adeno-associated virus 2 capsid protein VP1 having the sequence of SEQ ID NO. 1 (Table 2) (GenBank accession number of the corresponding nucleic acid sequence is J01901.1). The corresponding amino acid positions for other homologous adeno-associated virus serotypes are shown in Table 3.

The term homologous in the context of the present specification relates to nucleic acid or amino acid sequences derived from different serotypes of AAV. The corresponding positions of different adeno-associated virus serotypes are shown in Table 3.

The term transgene in the context of the present specification relates to a gene or genetic material that has been transferred from one organism to another. In the present context, the term may also refer to transfer of the natural or physiologically intact variant of a genetic sequence into tissue of a patient where it is missing. It may further refer to transfer of a natural encoded sequence the expression of which is driven by a promoter absent or silenced in the targeted tissue.

The term recombinant in the context of the present specification relates to a nucleic acid, which is the product of one or several steps of cloning, restriction and/or ligation and which is different from the naturally occurring nucleic acid. A recombinant virus particle comprises a recombinant nucleic acid.

The term intravitreal administration in the context of the present specification relates to a route of administration of a pharmaceutical agent, for example a viral vector, in which the agent is delivered into the vitreous body of the eye. Intravitreal administration is a procedure to place a medication directly into the space in the back of the eye called the vitreous cavity, which is filled with a jelly-like fluid called the vitreous humour gel.

The term subretinal administration in the context of the present specification relates to a route of administration of a pharmaceutical agent, particularly a viral vector in the context of this specification, into the space between retinal pigment epithelium (RPE) cells and photoreceptors.

The term polypeptide in the context of the present specification relates to a molecule consisting of 50 or more amino acids that form a linear chain wherein the amino acids are connected by peptide bonds. The amino acid sequence of a polypeptide may represent the amino acid sequence of a whole (as found physiologically) protein or fragments thereof. The term “polypeptides” and “protein” are used interchangeably herein and include proteins and fragments thereof. Polypeptides are disclosed herein as amino acid residue sequences.

Amino acid residue sequences are given from amino to carboxyl terminus. Capital letters for sequence positions refer to L-amino acids in the one-letter code (Stryer, Biochemistry, 3^{rd} ed. p. 21). Lower case letters for amino acid sequence positions refer to the corresponding D- or (2R)-amino acids. Sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

The term variant refers to a polypeptide that differs from a reference polypeptide, but retains essential properties. A typical variant of a polypeptide differs in its primary amino acid sequence from another polypeptide used as reference. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide may be naturally occurring such as an allelic variant, or it may be a variant that is not known to occur naturally.

The term biological activity in the context of the present specification relates to a specifically measurable quantity displayed by certain variants of the polypeptides enclosed herein. For example, in the context of a viral vector's ability to facilitate gene transfer into a retinal cell, the biological activity of a capsid variant may be assayed by measuring expression of a transgene (e.g. firefly luciferase) into cultured cells or into a model organism in-vivo in a standard assay.

In the context of the present specification, the terms sequence identity and percentage of sequence identity refer to the values determined by comparing two aligned sequences. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless stated otherwise, sequence identity values provided herein refer to the value obtained using the BLAST suite of programs (Altschul et al., J. Mol. Biol. 215:403-410 (1990)) using the above identified default parameters for protein and nucleic acid comparison, respectively.

In the context of the present specification, the term amino acid linker refers to an oligopeptide of variable length that is used to connect two polypeptides in order to generate a single chain polypeptide. Exemplary embodiments of linkers useful for practicing the invention specified herein are oligopeptide chains consisting of 1 to 6 amino acids. A non-limiting example of an amino acid linker is AAA or AA, as exemplified below.

AAV is a small non-pathogenic virus that infects humans and other primate species.

The AAV2 infection starts by docking to the cell surface receptor heparan sulphate proteoglycan (HSPG). Its low-affinity binding to glycans induces a reversible structural rearrangement of the capsid that promotes binding to the co-receptor αvβ65 or α5β1 integrin inducing formation of a clathrin-coated pit. The clathrin-coated pit becomes internalized via endocytosis and the viral particles are transported to the nucleus. The pH drops due to acidification of the endosomal compartments, which is a feature of the endosomal vesicle maturation. Acidification-triggered conformational change takes place in the capsid, and the virus escapes from the late endosome by lipolytic pore formation.

In wild-type AAV the genome is built of a 4.7 kilobase long single stranded DNA (ssDNA), either positive- or negative-sensed. The genome comprises three open reading frames (ORFs) flanked by inverted terminal repeats (ITRs). The ITRs are self-complementary, CG-rich, T-shaped hairpins at the 5′ and 3′-end of the AAV genome and the only necessary viral component present in recombinant vector genomes. The ITR include a terminal resolution site (TRS) and a Rep binding element (RBE), which facilitate replication and encapsidation of the viral genome. The ORFs encode the genes rep, cap, AAP. Four multifunctional non-structural Rep proteins encoded by rep are required for the AAV life cycle. Cap encodes the capsid proteins VP1, VP2 and VP3, which interact together to form a capsid of an icosahedral symmetry, and the assembly-activating protein (AAP), which is required for stabilizing and transporting newly produced VP proteins from the cytoplasm into the cell nucleus. All three VPs are translated from one mRNA and spliced differently. The largest 90 kDa VP1 is an unspliced transcript, the 72 kDa VP2 is translated from a non-conventional ACG start codon whereas the smallest 60 kDa VP3 is translated from an AUG codon. All the three VPs have overlapping C-termini.

The VP3 constitutes 90% of the capsid and VP1 and VP2 share a common C-terminal amino acid sequence with VP3 but have N-terminal extensions that stay buried inside the capsid. The VP1 unique N-terminal sequence contains phospholipase A2 (PLA2) activity that is required for infection, and nuclear localization signals. The three VP monomers assemble into two-fold, three-fold, and five-fold axes of symmetry to create the 60-subunit of AAV capsid. A first aspect of the invention relates to an adeno-associated virus (AAV) capsid polypeptide comprising a peptide insert at a peak or a spiky protrusion at position 453 or 587 of the AAV serotype 2 capsid. The peptide insert is selected from the following sequences:

(Cap3; SEQ ID NO 10)
SASEAST,
(Cap5; SEQ ID NO 11)
DTRPHDQ,
(Cap7; SEQ ID NO 12)
EHYNSTC,
(Cap9; SEQ ID NO 13)
PNPNCTL,
(Cap11; SEQ ID NO 14)
TPPSITA,
(Cap12; SEQ ID NO 15)
CGESSYL,
(Cap13; SEQ ID NO 16)
PRTPHTA
and
(Cap14; SEQ ID NO 17)
ELCDGFA,

particularly the insert is selected from SASEAST (Cap3; SEQ ID NO 10), TPPSITA (Cap11; SEQ ID NO 14), PRTPHTA (Cap13; SEQ ID NO 16) and ELCDGFA (Cap14; SEQ ID NO 17).

The peptide insert as laid out above may be comprised in a 7-13mer consisting of one of the above sequence, flanked by 0-6 linker amino acids (6 being the maximum number of the total of N and C terminally flanking amino acids) selected from, but not limited to, Ala, Leu, Gly, Ser, and Thr inserted N- and/or C-terminally of the insert sequences given in the preceding paragraph.

Any position given for AAV2 sequences given herein is in relation to the reference sequence accessible at GenBank entry No. J01901.1 (Adeno-associated virus 2, complete genome). The corresponding amino acid sequence is given as SEQ ID NO 1 in table 2.

Spiky protrusions (peaks) represent the most exposed regions of the capsids. The highest peak is located at amino acid position 453 and second highest at position 587 on the AAV2 capsid. These peaks accept peptide insertions without disturbing capsid assembly and provide opportunities for targeting non-permissive cells. Likewise, protrusions represent critical sites of AAVs host interaction, receptor binding and immunogenicity. Of note, in certain embodiments, R585 and 588 are mutated to attain optimal efficiency if the insert is inserted at position 453.

When referring to a certain peak or a spiky protrusion position, where the peptide insert is introduced, it is to be understood that the peptide insert may be directly after (in C-terminal direction) this position or one to six amino acids further in C-terminal direction. For example, the peptide insert defined to be at position 587 (an insertion defined as between the pre-insertion positions 587 and 588) may start at position 588 or 589 (one or two amino acids further in C-terminal direction) or 590 (three amino acids further in C-terminal direction). All of these insert positions allow for peptide inserts that do not disturb the overall capsid structure, but rather may increase transduction efficacy.

This peptide insert increases the retinal penetration and transduction efficacy of the virus after intravitreal delivery.

In certain embodiments, the peptide insert is selected from SASEAST (Cap3; SEQ ID NO 10), DTRPHDQ (Cap5; SEQ ID NO 11), EHYNSTC (Cap7; SEQ ID NO 12), PNPNCTL (Cap9; SEQ ID NO 13), TPPSITA (Cap11; SEQ ID NO 14), CGESSYL (Cap12; SEQ ID NO 15), PRTPHTA (Cap13; SEQ ID NO 16) and ELCDGFA (Cap14; SEQ ID NO 17), In certain embodiments, the peptide insert is selected from SASEAST (Cap3; SEQ ID NO 10), TPPSITA (Cap11; SEQ ID NO 14), PRTPHTA (Cap13; SEQ ID NO 16) and ELCDGFA (Cap14; SEQ ID NO 17).

The peptide insert can be present in the AAV capsid polypeptide as is or in the context of 1, 2, 3 or 4 flanking spacer amino acids at the amino terminus and/or at the carboxyl terminus.

Suitable spacer AAs include, but are not limited to alanine, leucine, glycine, threonine and serine.

In the inventors' examples, due to the cloning strategy used, the insertion between N₅₈₇ and R₅₈₈ of the AAV2 VP1 is a 12mer of the form R₅₈₅G N A A A X₁ X₂ X₃ X₄ X₅ X₆ Xr-A A R₅₈₈ Q A A, whereas X₁-X₇ represents the inserted heptamer oligo and A the flanking linkers. It is to be understood that this is an example only and the invention is not limited to inserts exactly at this position having exactly these flanking sequences.

In embodiments relating to an inserting position designated 587 in AAV2, the oligo is always inserted between positions 587 and 588 on VP1 of AAV2. It is an insertion, so the VP1 AAV2 numbering does not change and subsequent positions are counted “without the insert” with respect to substitutions and the like. The inserted peptide contains the heptamer as specified herein, but may potentially also be shorter to function or longer. In embodiments represented in the examples, the inventors inserted a 12-mer in the form of AAA-X1X2X3X4X5X7-AA, whereas the linker amino acids can be freely selected from Ala, Leu, Gly, Ser, Thr. The N- and C-terminal linkers can be of 0-5 AA length. In certain embodiments, an insertion peptide can be of 5-13 amino acids length, comprising 0-6 linker amino acids.

In certain embodiments, the peptide insertion (elsewhere in this specification referred to as “oligo”) can move 1-5AA further to the C-terminus. For AAV2, the insertion site can also be at (e.g., immediately C-terminal to) amino acid 453.

According to an alternative of this first aspect of the invention, any of the peptide sequences recited above are inserted at position 453 of SEQ ID NO 001 instead of position 587. Again, the position of insertion may vary and the peptide sequence may be flanked by inserts as discussed above and may consist of 5-13 AAs. In addition to the peptide insertion at position 453, R587A and R588A mutations may be introduced into VP1 of AAV2 (Boucas Jet al. J Gene Med. 2009 December; 11(12):1103-13. doi: 10.1002/jgm.1392) According to another alternative of this aspect of the invention, a position homologous to position 587/588 or 453 in AAV serotype 2 can be selected (Table 3: #1, #2) to construct an AAV of another serotype with one of the inserted peptide sequences recited above.

In certain embodiments, the AAV capsid protein is characterized by one or several tyrosine to phenylalanine substitutions of tyrosine residues, wherein the tyrosine residues occur in the wild-type capsid sequence at positions 252, 272, 444, 500, 700, 704 and 730.

Certain of the capsid variants herein were selected by evolutionary methods in the context of a tyrosine-modified capsid.

In certain embodiments, the AAV capsid protein is characterized by one or several tyrosine to phenylalanine substitutions at positions 252, 272, 444, 500, 704 and 730.

In certain even more particular embodiments, the AAV capsid protein is characterized by several tyrosine to phenylalanine substitutions at all of the positions 252, 272, 444, 500, 700 and 730.

According to another alternative of this aspect of the invention, a tyrosine position homologous to any of the positions 252, 272, 444, 500, 700, 704 and 730 in AAV serotype 2 can be selected for substitution to phenylalanine (Table 3: #3-#9) to construct an AAV of another serotype with tyrosine to phenylalanine substitutions. Also, other tyrosine positions may be substituted for phenylalanine.

In certain embodiments, the AAV capsid protein is characterized by one or several threonine to valine substitutions, particularly T491V.

According to another alternative of this aspect of the invention, a threonine position homologous to the position 491 in AAV serotype 2 can be selected for substitution to valine (Table 3: #10) to construct an AAV of another serotype with a threonine to valine substitution.

In certain embodiments, the tyrosine to phenylalanine substitutions mentioned above are combined with the threonine to valine substitutions, particularly T491V.

Y-F and T-V mutations decrease ubiquitination and thus proteasomal degradation of the viral particle once internalized by the cell, thereby increasing transduction efficacy.

In certain embodiments, the adeno-associated virus capsid polypeptide comprises the amino acid sequence selected from SEQ ID NO 2-SEQ ID NO 9, or a sequence having at least 85% identity thereto and at least 90% of the biological activity of a sequence selected from SEQ ID NO 2-SEQ ID NO 9.

### Biological Activity Assay:

Wherever reference is made herein to a polypeptide “having at least a certain percentage of the biological activity of a (reference) sequence”, this biological activity can be measured as follows:

HEK293 cells are co-transfected with a plasmid encoding the test or the reference polypeptide, the plasmid harbouring helper adenoviral genes and a transgene plasmid, selected from scCMV-mCitrine, scEf1a-mCitrine, scCMV-EGFP, scEf1a-EGFP as a reporter, using the calcium-phosphate precipitation method. Vectors containing the transgene are concentrated by density purification over an iodixanol gradient (Axis-Shield, Oslo) and the 40% iodixanol fraction subsequently buffer exchanged by for example amicon filtration (Millipore). The AAV fraction is titered for DNase-resistant vector genomes by real-time PCR relative to a standard vector. 3 μl of viral vector are intravitreally injected into anaesthetized (100 mg/kg ketamine and 10 mg/kg xylazine) wild type C57BL/6J mice. For injections, the virus is titer-matched to E11 GC/ml and 3 ul thereof is injected—i.e. 3×E8 vg per eye. Mouse retinas are removed 3 weeks post-injection for immunohistochemical analysis. Mouse eyes retinal explants (7 days post-transduction) are fixed with 4% (wt/vol) paraformaldehyde in PBS for 40 min at room temperature (RT), cryoprotected over three consecutive nights at 4° C. in graded sucrose solutions (10%, 20%, and 30% sucrose in PBS), embedded in cryomolds with O.C.T. compound (Sakura Finetek), and frozen in liquid nitrogen-cooled 2-methylbutane. Vertical sections of 10 μm thickness are cut on a cryostat, mounted on SuperFrost glass slides (Menzel) and immunohistochemically labelled with suitable primary and secondary antibodies to detect EGFP, YFP, mCitrine or Turbo635 fluorescence as well as specific retinal cell types. The reporter expression in the target cell type is quantified, typically by fluorescence, and compared to the amount of reporter in the remainder of the tissue to assess the biological activity of the engineered virus capsid. Another measure for biological activity of the engineered virus capsid is the percentage of target cells transduced and the area of tissue expressing. For the avoidance of doubt, the assay quantifying reporter expression in the target cell type and comparing to the amount of reporter in the remainder of the tissue is employed when measuring the biological activity.

A second aspect of the invention relates to a nucleic acid sequence encoding the AAV capsid polypeptide according to the first aspect.

In particular embodiments, the AAV sequence according to the invention does not contain a Rep element required for integration into a host genome. Non-integrating viruses are safer for application in humans. In certain embodiments, the nucleic acid sequence is designed according to the self-complementary AAV vector genome concept. In all cases a single stranded DNA sequence is used.

In certain embodiments, the nucleic acid sequence comprises a transgene with or without regulatory sequences.

In certain embodiments, the transgene encodes the sequence of a correct protein (i.e. RPE65), a siRNA or shRNA (designed to target regions of mRNA to degrade wild-type and mutant RNA of dominant disease-causing genes), or a CRISPR/Cas-gRNA cassette for gene editing.

In certain embodiments, the transgene encodes a light-sensitive protein, such as an invertebrate or vertebrate opsin or a variant thereof.

In certain embodiments, the transgene encodes channelrhodopsin-2 or a variant thereof.

In certain embodiments, the transgene encodes a microbial light-gated inhibitory ion pump, such as for example halorhodopsin (eNpHR) or archaerhodopsin (ArchT).

In certain embodiments, the transgene encodes a GPCR opsin such as for example human rhodopsin, melanopsin or a cone opsin.

In certain embodiments, the transgene encodes an acceptor protein for a photo-switchable ligand.

In certain embodiments, the transgene is under control of a promoter sequence operable in a mammalian cell. In certain embodiments, the promoter sequence is operable in a human retinal cell.

In certain embodiments, the promoter is a ubiquitous promoter. In certain embodiments, the promoter is a cell-specific promoter.

In certain embodiments, the promoter is a CMV immediate early promoter. In certain embodiments, the promoter is a human Ef1a promoter. In certain embodiments, the promoter is a photoreceptor specific promoter.

A third aspect of the invention relates to an agent selected from the AAV capsid polypeptide according to the first aspect, an AAV vector comprising a capsid polypeptide according to the first aspect, and a nucleic acid sequence according to the second aspect, for use in medicine/as a medicament.

In certain embodiments, AAV vectors are used to transduce cells of the eye.

The eye is made up of three layers, composed of various anatomical structures. The fibrous tunic is the outermost layer and is composed of the cornea and sclera. The vascular tunic or uvea is the middle layer and consists of the choroid, ciliary body, pigmented epithelium and the iris. The retina is the innermost layer, which gets its oxygenation from the blood vessels of the choroid (posteriorly) as well as the retinal vessels (anteriorly).

The spaces between the cornea and lens are filled with the aqueous humour, and the entire posterior cavity behind the lens is filled with the vitreous body, a jelly-like substance. The vitreous body is composed of water, collagen, fibrils, hyaluronic acid and ions. The void spaces in the retina that are not occupied by neurons or blood vessels; are filled by the processes of Müller glial cells that span all the retinal cell layers.

The outer limiting membrane (OLM) of the retina is formed from junctions between Müller cells (MCs) and inner segments of photoreceptor cells and acts as a metabolic barrier between the subretinal space, restricting passage of large molecules. The inner limiting membrane (ILM) of the retina is formed by lateral connection between MCs end-feet and basement membranes and acts as a diffusion barrier between the vitreous humor and the neural retina.

The retina is composed of macula, optic disc, fovea and peripheral retina. A photoreceptor cell is a specialized type of neuroepithelial cell found in the retina. Three types of photoreceptor cells are known: rods, cones, and photosensitive retinal ganglion cells. The rods are distributed at the peripheral region of the retina; whereas the central pigmented region called macula is enriched for cone photoreceptor cells. The retinal pigment epithelium (RPE) provides nutrition and maintains the health of photoreceptor cells. The nuclei of photoreceptor cells constitute the outer nuclear layer (ONL) whereas the nuclei of bipolar, amacrine and horizontal cells are located in the inner nuclear layer (INL).

In certain embodiments, the agent selected from the AAV capsid polypeptide according to the first aspect, an AAV vector comprising a capsid polypeptide according to the first aspect, and a nucleic acid sequence according to the second aspect, is for use in treatment of a condition affecting
a. a retinal or retinal pigment epithelium cell and/or
b. a photoreceptor, a bipolar cell, an amacrine cell or a ganglion cell of the retina.

In certain embodiments, the agent is for use in treatment of glaucoma, retinitis pigmentosa, macular degeneration, retinoschisis, Leber's Congenital Amaurosis, diabetic retinopathy, achromatopsia, or color blindness, melanoma-associated retinopathy, congenital stationary night blindness, cone-rod dystrophy, late stage age-related macular degeneration, maculopathies, early onset severe retinal dystrophy, achromatopsia, ocular albinism, oculocutaneous albinism, Stargardt disease, choroideremia, Spinocerebellar Ataxia type 7 (SCAT), lysosomal storage diseases that affect the cornea, such as Mucopolysaccharidosis (MPS) IV and MPS VII, retinoblastoma, ocular melanoma, hypertensive retinopathy.

In certain embodiments, the agent of the invention can be employed for treatment or prevention of a disease affecting the inner ear.

In certain embodiments, the agent is administered by
a. intravitreal administration, particularly by intravitreal injection, or by
b. subretinal administration.

In certain embodiments, the agent is delivered to the posterior segment, the anterior segment, the sclera, the choroid, the conjunctiva, the iris, the lens, or the cornea.

Similarly, within the scope of the present invention is a method or treating a condition selected from rod-cone dystrophies including retinitis pigmentosa, cone-rod dystrophies including macular degeneration and congenital stationary night blindness (CSBN1) in a patient in need thereof, comprising administering to the patient a viral vector comprising an AAV capsid and/or a nucleic acid sequence according to the above description.

Similarly, a dosage form for the prevention or treatment of a condition selected from cone-rod dystrophies, rod-cone dystrophies and congenital stationary night blindness is provided, comprising an AAV capsid and/or a nucleic acid sequence according to one of the above aspects of the invention.

The agents and methods disclosed herein do also provide significant advantages in early disease stages when damage to PR is small, as the vectors facilitated by the invention are more efficient than any existing alternatives.

Wherever alternatives for single separable features such as, for example, an AAV serotype protein or capsid peptide insert sequence or medical indication are laid out herein as “embodiments”, it is to be understood that such alternatives may be combined freely to form discrete embodiments of the invention disclosed herein.

The invention is further illustrated by the following examples and figures, from which further embodiments and advantages can be drawn. These examples are meant to illustrate the invention but not to limit its scope.

### DESCRIPTION OF THE FIGURES

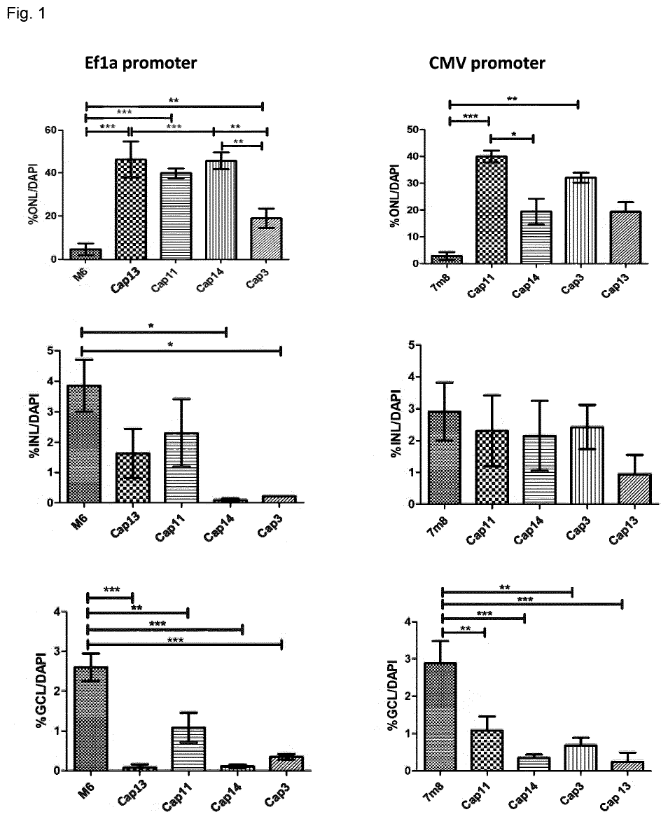

FIG. 1 Cell specificity of expression for selected capsid variants compared to the selection backbone AAV2 (Y252, 272, 444, 500, 700, 730F), referred to in the following as AAV2(M6), and the state-of-the-art synthetic capsid AAV2(7m8). All AAVs have been packaged as self-complementary (scAAVs) expressing either eGFP or mCitrine under two ubiquitous promoters hEF1a and CMV in order to remove the possible bias of one promoter towards a certain retinal cell type. Expression in photoreceptors (ONL) is significantly enhanced for the novel variants after intravitreal injection as opposed to AAV2(M6) and AAV2(7m8). 2.5 μl injected intravitreally, titer matched to 1E+11 vg/ml (except Cap14 at 3E+10). 4 retinas counted, mean±s.d. INL, inner nuclear layer; GCL, ganglion cell layer; DAPI, number of all cell bodies stained with the nuclear stain DAPI.

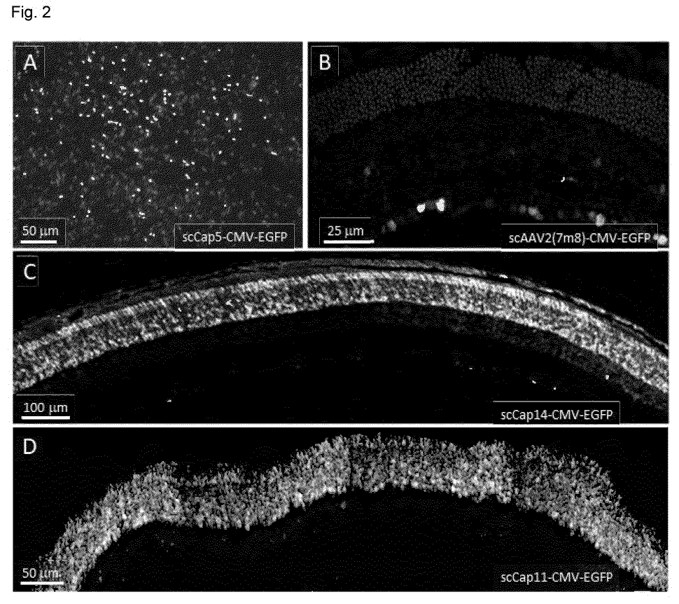

FIG. 2 Panretinal strong expression throughout the ONL for novel capsid variants as indicated at 100× reduced functional titer compared to state-of-the art AAV2(7m8). All injections were titer-matched and 7.4E+7 vg delivered into the vitreous of C57BL/6 mice. A. Low magnification picture focused on the ONL of a mouse retinal whole mount after transduction with scCap5-CMV-EGFP. B-C. Vertical retinal cryosection. From the photomicrograpsh the potency of the novel capsids at low titer compared to AAV2(7m8) is obvious.

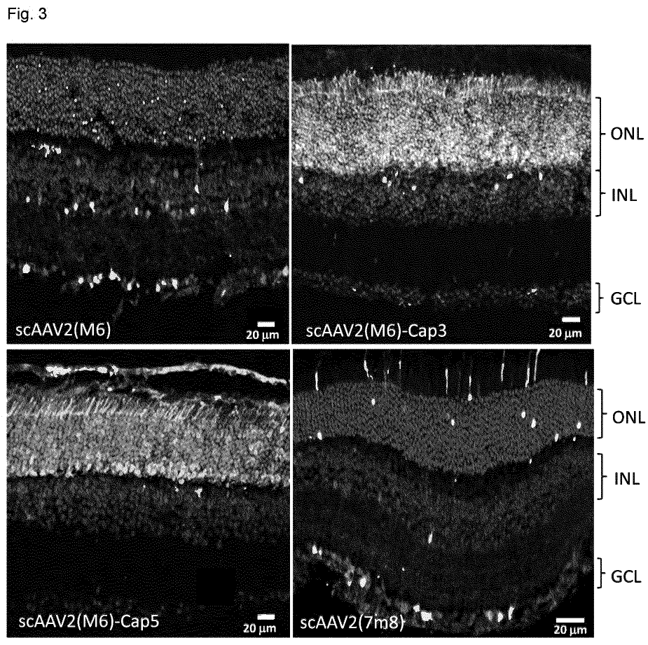

FIG. 3 Comparison of transduction after intravitreal injection of 2.5 μl scAAV into C57BL/6 mouse eyes (as described e.g. in van Wyk et al., PLoS Biol, 2015. 13(5): p. e1002143). mCitrine expression under control of the human Ef1a promoter, titers matched to 1E+11 vg/ml. From the photomicrographs of vertical and immunolabeled retinal cryosections it is obvious that scAAV2(Cap3) and scAAV2(Cap5) penetrate better through the ILM and retinal layers and show an unparalleled expression efficacy in the photoreceptors of the ONL when compared to state-of-the art AAV(7m8) or the crude library backbone AAV2(M6).

### EXAMPLES

### Example 1: Generation of Peptide Display Library

The AAV capsid is an icosahedron with 60 subunits. At the 3-fold axes of the AAV capsid, which are formed when the capsid subunits assemble, spiky protrusions (peaks) are formed, that represent the most exposed regions of the capsid. On the AAV2 capsid, the highest peak is located at amino acid (AA) position 453 and the second highest at AA positions 587/588 (AAV2-VP1 numbering). These peaks accept peptide insertions without disturbing the viral capsid assembly and represent critical sites of host interaction, receptor binding and immunogenicity. To generate a peptide display library the inventors inserted a randomized heptapeptide between N₅₈₇ and R₅₈₈ of the open reading frame of VP1 of AAV2 containing six additional tyrosine to phenylalanine mutations (Y252,272,444, 500, 700, 730F), referred to as AAV2(M6) (Table 2). Y-F and T-V mutations (Buning & Srivastava, Mol Ther Methods Clin Dev. 2019 Jan. 26; 12:248-265. doi: 10.1016/j.omtm.2019.01.008) were previously shown to decrease ubiquitination and thus proteasomal degradation of the viral particle once internalized by the cell (Petrs-Silva et al., Mol Ther, 2011. 19: p. 293-301.) thereby increasing transduction efficacy and potentially favoring the selection process in an in vivo evolution where variants are in the initial rounds represented at very low counts. Random insertions can be made at homologous sites in the GH loops (loop IV) of other capsid serotypes, as shown in Table 3. The approach on how the inventors developed the library was adopted from Perabo and colleagues (Perabo et al., Mol Ther, 2003. 8: p. 151-157). Two unique restriction sites, AscI and NotI, were introduced between amino acids 587 and 588 of the AAV2(M6) genome (Table 2). The introduced DNA fragment forming the AscI and NotI sites encoded a stop codon flanked by alanine linkers (AAAstopAA). Next, a single-stranded randomized 7mer pool with NNB codons was synthesized as

(SEQ ID NO 25)
5′-TTGGCGCGCCGCVNNVNNVNNVNNVNNVNNVNNGGCGGCCGCTTTTT
TCCTTGA-3′

(Eurofins Genomics) and converted into a pool of dsDNA fragments by second-strand synthesis using the antisense primer 5′-CTCAAGGAAAAAAGC-3′ (SEQ ID NO 26). To generate the 7mer display library, the dsDNA random oligonucleotides were cloned into the AscI-NotI site of the modified AAV(M6) genome thereby replacing the stop codon. The virus library was generated such that each viral genome was packaged or encapsidated within the capsid protein variant which that genome encoded (genotype-phenotype coupling). In addition, all capsids were produced in a way that only one kind of peptide was present in each of the 60 subunits. In this way, functional improvements identified through selection can be linked to the genome sequence encoding this improved function contained within the viral capsid.

### Example 2: Selection of Peptide Variants

This library was subjected to positive selection within C57BL/6_Opto-mGluR6 mice, generated by our laboratory (van Wyk et al., PLoS Biol, 2015. 13(5): p. e1002143), expressing the red fluorescent marker FP635 in retinal ON-bipolar cells. This mouse line was selected based on the rationale that ON-bipolar cells are located deep within the retina and are known not to be permissive to AAV transduction, so that the selection would favor well penetrating AAVs and variants with advantageous properties to transduce ON-bipolar cells. In brief, 5-8 transgenic mice of 4-6 weeks of age were injected intravitreally with 2.5 μl of iodixanol-purified library with a genomic titer of approximately 5×10¹¹ viral genomes (vg)/ml into both eyes as described in (van Wyk et al., PLoS Biol, 2015. 13(5): p. e1002143; van Wyk et al., Front Neurosci, 2017. 11: p. 161).

After 10 days, eyes were enucleated and retina dissociated using a light papain protease treatment, followed by fluorescence activated cell sorting (FACS) of the ON-bipolar cell population. Successful virions were then PCR-amplified from DNA extractions and further cloned and repackaged for subsequent rounds of injections.

At each cloning step of 7mer oligos into the AAV2(M6) genome, four parallel ligations were run and 12 clones of each pool plated and sequenced to estimate convergence of the library after each in vivo selection step. This process of in vivo directed evolution created ON-bipolar cell permissive AAV variants through the application of positive selection, similar to the process of natural evolution.

From the in vivo selected library the capsid genes of 81738 variants were identified by next-generation sequencing. 15 heptapeptide sequences from the top 67 ranked were selected for functional evaluation and characterization as outlined in table 1, on a combination of high overall transduction efficacy (accumulation in NGS), a preference for ON-bipolar cell targeting and motifs that either appeared early in the selection rounds or that looked promising in terms of their amino acid sequence (i.e. containing negatively charged residues or proline residues). A key characteristic of all library variants is the separation of the positive charges (R₅₈₅ and R₅₈₈) of the heparan sulphate proteoglycan binding motif of AAV2 through the insertion. However, the peptide sequence together with the two alanine of the linker sequence can re-install the ability to bind to heparan sulphate proteoglycan (Uhrig . . . Bu\ddot{n}ing, Gene Therapy). Among the 7mer insert sequences, there were moderate preferences at particular positions, e.g. charged amino acids at position 1 or 2 and/or at position 5 or 6 and a polar amino acid, such as Cys or Ala at position 7 (Table 1).

### Example 3: Evaluation of Novel Capsid Variants

A recombinant form of AAV2(M6)-7mer ˜ was cloned for all 15 selected peptides as of table 1. The inventors packaged with a scCMV-EGFP transgene (CMV: cytomegalovirus promoter). Except for two variants (Cap8 and Cap10), all capsids packaged well with titers between 10¹¹-10¹² vg/ml. Three weeks following intravitreal injections into adult mice, robust expression primarily in photoreceptors was observed for Cap3, Cap5, Cap7, Cap9, Cap 11, Cap 13 and Cap 14 (Table 1, FIGS. 1-3), but also some expression in cells of the inner retina such as bipolar cells, amacrine cells and Müller cells and very few retinal ganglion cells. In a direct comparison with the performance of AAV2(7m8), which is also an evolved AAV2 variant (Dalkara et al., Sci Transl Med, 2013. 5: p. 189ra76.) and currently considered state-of-the art for intravitreal injections, the inventors' novel variants penetrated significantly better though the retina and transduced significantly more photoreceptors and significantly less ganglion cells (FIG. 1, FIGS. 1-3). The above results were confirmed by packaging the 8 pre-selected variants (Cap3, Cap5, Cap7, Cap9 Cap 11, Cap 13 and Cap 14; Table 1) also with the schEf1a-EGFP-mCitrine transgene (hEfla: human Ef1a promoter, “strong” native mammalian ubiquitous promoter) and injecting adult mice intravitreally with a matched titer of 1×10¹¹ vg/ml.

Equivalently, the new variants transduced photoreceptors in significantly larger number than the titer-matched controls AAV2(M6) and AAV(7m8). Consequently, the selected variants appear to possess superior inner limiting membrane and retinal penetration properties compared to AAV2(M6) and AAV2(7m8) and are significantly more effective in transducing the photoreceptors in the outer nuclear layer (ONL) (FIG. 1).

Cap14 packaged with scCMV-EGFP and schEf1a-EGFP and injected at 3E+10 vg/ml performed significantly better than AAV2(7m8) injected at the same low dose (FIGS. 1, 2) and even better than AAV2(7m8) at the minimal dose typically used in mouse of 1E+12 vg/ml. This is promising in light of using lower doses in patients to reduce concerns of toxicity and immune responses.

TABLE 1
Next-generation sequencing of ON-bipolar cell isolated variants from directed
evolution revealed a high degree of convergence in viral libraries. The7mer
inserts were flanked by alanine spacers in the form of ~N₅₈₇AAASASEASTAA
R₅₈₈~ (SEQ ID NO 29) here exemplified for variant #3.
ON-Bipolar cell dominance: =occurrence [ON-bipolar cells / Non-On-bipolar cells]

| | | ON- bipolar cell | | Non-ON-bipolar | | ON-bipolar | |
|---|---|---|---|---|---|---|---|
| | Variant | fraction | | cell fraction | | cell | |
| Clone | (Cap) # | F | O (%) | F | O (%) | dominance | SEQ ID NO |
| HHNDRAP^{#} | 15 | 6050 | 9.9 | 1121 | 5.4 | 1.8 | 18 |
| DGNLHKS^{#} | 2 | 2837 | 4.7 | 1069 | 5.1 | 0.9 | 19 |
| SASEAST* | 3 | 2814 | 4.6 | 1511 | 7.2 | 0.6 | 10 |
| HSDSSKP | 4 | 1583 | 2.6 | 370 | 1.8 | 1.5 | 20 |
| DTRPHDQ | 5 | 1378 | 2.3 | 473 | 2.3 | 1.0 | 11 |
| PNPNCTL | 9 | 403 | 0.7 | 41 | 0.2 | 3.4 | 13 |
| TLTGLCP~ | 8 | 505 | 0.8 | 50 | 0.2 | 3.5 | 21 |
| EHYNSTC | 7 | 310 | 0.5 | 27 | 0.1 | 3.9 | 12 |
| NHAPNHC | 6 | 292 | 0.5 | 25 | 1 | 4.0 | 22 |
| DRRPGIC | 1 | 209 | 0.34 | 4 | 0.02 | 17.9 | 23 |
| VDATCMQ~ | 10 | 402 | 0.7 | 40 | 0.2 | 3.4 | 24 |
| TPPSITA* | 11 | 342 | 0.6 | 35 | 0.2 | 3.3 | 14 |
| CGESSYL | 12 | 334 | 0.5 | 37 | 0.2 | 3.1 | 15 |
| PRTPHTA* | 13 | 227 | 0.4 | 27 | 0.1 | 2.9 | 16 |
| ELCDGFA* | 14 | 222 | 0.4 | 38 | 0.2 | 2.0 | 17 |

F = Frequency;
O = Occurrence
^{#}appeared in 2^{nd} selection round
*top candidates selected from in vivo screening
~Capsids with inserts Nr. 8 and Nr. 10 did not package

### Positively Charged

### Negatively Charged

All 15 selected clones were ranked in the top 67 of 60′884 peptides sequenced from the isolated ON-bipolar cell fractions of C57BL/6_Opto-mGluR6 mice (van Wyk et al., PLoS Biol, 2015. 13(5): p. e1002143).

TABLE 2
Amino acid sequences of AAV2 capsid protein VP1 with different insertions.
VP3 (italicized sequence) overlapping with VP1, tyrosine to phenylalanine (Y-F) mutations
are underlined, amino acid numbering refers to the whole VP1 sequence. The highest peak
at G₄₅₃ and the second highest peak at N587, where the random 7mers were inserted, are
bold and underlined. The insertion is boxed, including the alanine linkers.
SI: SEQ ID NO of the peptide insertion sequence; FS: SEQ ID NO of the full capsid
sequence

| SI | Insertion | FS | Sequence |
|---|---|---|---|
| — | AAV2 | 1 | MAADGYLPDWLEDTLSEGIRQWWKLKPGPPPPKPAERHKDDSR |
| | (Y252, | | GLVLPGYKYLGPFNGLDKGEPVNEADAAALEHDKAYDROLDSG |
| | 272, 444, | | DNPYLKYNHADAEFQERLKEDTSFGGNLGRAVFQAKKRVLEPL |
| | 500, 700, | | GLVEEPVKTAPGKKRPVEHSPVEPDSSSGTGKAGQQPARKRLN |
| | 730F) | | FGQTGDADSVPDPQPLGQPPAAPSGLGTNTMATGSGAPMADNN |
| | | | EGADGVGNSSGNWHCDSTWMGDRVITTSTRTWALPTFNNHLYK |
| | | | QISSQSGASNDNHFFGYSTPWGYFDFNRFHCHFSPRDWORLIN |
| | | | NNWGFRPKRLNFKLFNIQVKEVTQNDGTTTIANNLTSTVQVET |
| | | | DSEYQLPYVLGSAHQGCLPPFPADVFMVPQYGYLTLNNGSQAV |
| | | | GRSSFYCLEYFPSQMLRTGNNFTFSYTFEDVPFHSSYAHSQSL |
| | | | DRLMNPLIDQYLYFLSRTNTPSGTTTQSRLOFSQAGASDIRDQ |
| | | | SRNWLPGPCYRQQRVSKTSADNNNSEFSWTGATKYHLNGRDSL |
| | | | VNPGPAMASHKDDEEKFFPQSGVLIFGKQGSEKTNVDIEKVMI |
| | | | TDEEEIRTTNPVATEQYGSVSTNLQRGNRQAATADVNTQGVLP |
| | | | GMVWQDRDVYLQGPIWAKIPHTDGHFHPSPLMGGFGLKHPPPQ |
| | | | ILIKNTPVPANPSTTFSAAKFASFITQYSTGQVSVEIEWELQK |
| | | | ENSKRWNPEIQFTSNYNKSVNVDFTVDINGVYSEPRPIGTRF |
| | | | LTRNL |
| 10 | SASEAST | 2 | MAADGYLPDWLEDTLSEGIRQWWKLKPGPPPPKPAERHKDDSR |
| | | | GLVLPGYKYLGPFNGLDKGEPVNEADAAALEHDKAYDRQLDSG |
| | | | DNPYLKYNHADAEFQERLKEDTSFGGNLGRAVFQAKKRVLEPL |
| | | | GLVEEPVKTAPGKKRPVEHSPVEPDSSSGTGKAGQQPARKRLN |
| | | | FGQTGDADSVPDPQPLGQPPAAPSGLGTNTMATGSGAPMADNN |
| | | | EGADGVGNSSGNWHCDSTWMGDRVITTSTRTWALPTFNNHLYK |
| | | | QISSQSGASNDNHFFGYSTPWGYFDFNRFHCHFSPRDWORLIN |
| | | | NNWGFRPKRLNFKLFNIQVKEVTQNDGTTTIANNLTSTVQVFT |
| | | | DSEYQLPYVLGSAHQGCLPPFPADVFMVPQYGYLTLNNGSQAV |
| | | | GRSSFYCLEYFPSQMLRTGNNFTFSYTFEDVPFHSSYAHSQSL |
| | | | DRLMNPLIDQYLYFLSRTNTPSGTTTQSRLQFSQAGASDIRDQ |
| | | | SRNWLPGPCYRQQRVSKTSADNNNSEFSWTGATKYHLNGRDSL |
| | | | VNPGPAMASHKDDEEKFFPQSGVLIFGKQGSEKTNVDIEKVMI |
| | | | 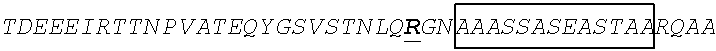 |
| | | | TADVNTQGVLPGMVWODRDVYLOGPIWAKIPHTDGHFHPSPLM |
| | | | GGFGLKHPPPQILIKNTPVPANPSTTFSAAKFASFITQYSTGQ |
| | | | VSVEIEWELQKENSKRWNPEIQFTSNYNKSVNVDFTVDINGVY |
| | | | SEPRPIGTRFLTRNL |
| 11 | DTRPHDQ | 3 | MAADGYLPDWLEDTLSEGIRQWWKLKPGPPPPKPAERHKDDSR |
| | | | GLVLPGYKYLGPFNGLDKGEPVNEADAAALEHDKAYDROLDSG |
| | | | DNPYLKYNHADAEFQERLKEDTSFGGNLGRAVFQAKKRVLEPL |
| | | | GLVEEPVKTAPGKKRPVEHSPVEPDSSSGTGKAGQQPARKRLN |
| | | | FGQTGDADSVPDPQPLGQPPAAPSGLGTNTMATGSGAPMADNN |
| | | | EGADGVGNSSGNWHCDSTWMGDRVITTSTRTWALPTFNNHLYK |
| | | | QISSQSGASNDNHFFGYSTPWGYFDFNRFHCHFSPRDWORLIN |
| | | | NNWGFRPKRLNFKLFNIQVKEVTQNDGTTTIANNLTSTVQVFT |
| | | | DSEYQLPYVLGSAHQGCLPPFPADVFMVPQYGYLTLNNGSQAV |
| | | | GRSSFYCLEYFPSQMLRTGNNFTFSYTFEDVPFHSSYAHSQSL |
| | | | DRLMNPLIDQYLYFLSRTNTPSGTTTOSRLQFSQAGASDIRDQ |
| | | | SRNWLPGPCYRQQRVSKTSADNNNSEFSWTGATKYHLNGRDSL |
| | | | VNPGPAMASHKDDEEKFFPQSGVLIFGKQGSEKTNVDIEKVMI |
| | | | 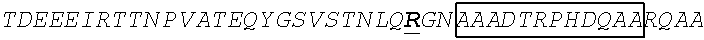 |
| | | | TADVNTQGVLPGMVWODRDVYLOGPIWAKIPHTDGHFHPSPLM |
| | | | GGFGLKHPPPQILIKNTPVPANPSTTFSAAKFASFITQYSTGO |
| | | | VSVEIEWELQKENSKRWNPEIQFTSNYNKSVNVDFTVDINGVY |
| | | | SEPRPIGTRFLTRNL |
| 12 | EHYNSTC | 4 | MAADGYLPDWLEDTLSEGIRQWWKLKPGPPPPKPAERHKDDSR |
| | | | GLVLPGYKYLGPFNGLDKGEPVNEADAAALEHDKAYDRQLDSG |
| | | | DNPYLKYNHADAEFQERLKEDTSFGGNLGRAVFQAKKRVLEPL |
| | | | GLVEEPVKTAPGKKRPVEHSPVEPDSSSGTGKAGQQPARKRLN |
| | | | FGQTGDADSVPDPQPLGQPPAAPSGLGTNTMATGSGAPMADNN |
| | | | EGADGVGNSSGNWHCDSTWMGDRVITTSTRTWALPTFNNHLYK |
| | | | QISSQSGASNDNHFFGYSTPWGYFDENRFHCHFSPRDWORLIN |
| | | | NNWGFRPKRLNFKLFNIQVKEVTONDGTTTIANNLTSTVQVFT |
| | | | DSEYQLPYVLGSAHQGCLPPFPADVFMVPQYGYLTLNNGSQAV |
| | | | GRSSFYCLEYFPSQMLRTGNNFTFSYTFEDVPFHSSYAHSQSL |
| | | | DRLMNPLIDQYLYFLSRTNTPSGTTTQSRLQFSQAGASDIRDQ |
| | | | SRNWLPGPCYRQQRVSKTSADNNNSEFSWTGATKYHLNGRDSL |
| | | | VNPGPAMASHKDDEEKFFPQSGVLIFGKQGSEKTNVDIEKVMI |
| | | | 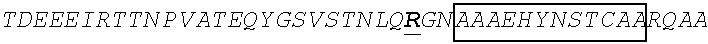 |
| | | | TADVNTQGVLPGMVWQDRDVYLQGPIWAKIPHTDGHFHPSPLM |
| | | | GGFGLKHPPPQILIKNTPVPANPSTTFSAAKFASFITQYSTGQ |
| | | | VSVEIEWELQKENSKRWNPEIQFTSNYNKSVNVDFTVDINGVY |
| | | | SEPRPIGTRFLTRNL |
| 13 | PNPNCTL | 5 | MAADGYLPDWLEDTLSEGIRQWWKLKPGPPPPKPAERHKDDSR |
| | | | GLVLPGYKYLGPENGLDKGEPVNEADAAALEHDKAYDRQLDSG |
| | | | DNPYLKYNHADAEFQERLKEDTSFGGNLGRAVFQAKKRVLEPL |
| | | | GLVEEPVKTAPGKKRPVEHSPVEPDSSSGTGKAGQQPARKRLN |
| | | | FGQTGDADSVPDPOPLGQPPAAPSGLGTNTMATGSGAPMADNN |
| | | | EGADGVGNSSGNWHCDSTWMGDRVITTSTRTWALPTFNNHLYK |
| | | | QISSQSGASNDNHFFGYSTPWGYFDENRFHCHFSPRDWQRLIN |
| | | | NNWGFRPKRLNFKLFNIQVKEVTQNDGTTTIANNLTSTVQVFT |
| | | | DSEYQLPYVLGSAHQGCLPPFPADVFMVPQYGYLTLNNGSQAV |
| | | | GRSSFYCLEYFPSQMLRTGNNFTFSYTFEDVPFHSSYAHSQSL |
| | | | DRLMNPLIDQYLYFLSRTNTPSGTTTQSRLQFSQAGASDIRDQ |
| | | | SRNWLPGPCYRQQRVSKTSADNNNSEFSWTGATKYHLNGRDSL |
| | | | VNPGPAMASHKDDEEKFFPQSGVLIFGKQGSEKTNVDIEKVMI |
| | | | 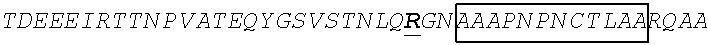 |
| | | | TADVNTQGVLPGMVWQDRDVYLQGPIWAKIPHTDGHFHPSPLM |
| | | | GGFGLKHPPPQILIKNTPVPANPSTTFSAAKFASFITQYSTGQ |
| | | | VSVEIEWELQKENSKRWNPEIQFTSNYNKSVNVDFTVDINGVY |
| | | | SEPRPIGTRFLTRNL |
| 14 | TPPSITA | 6 | MAADGYLPDWLEDTLSEGIRQWWKLKPGPPPPKPAERHKDDSR |
| | | | GLVLPGYKYLGPENGLDKGEPVNEADAAALEHDKAYDRQLDSG |
| | | | DNPYLKYNHADAEFQERLKEDTSFGGNLGRAVFQAKKRVLEPL |
| | | | GLVEEPVKTAPGKKRPVEHSPVEPDSSSGTGKAGQQPARKRLN |
| | | | FGQTGDADSVPDPQPLGQPPAAPSGLGTNTMATGSGAPMADNN |
| | | | EGADGVGNSSGNWHCDSTWMGDRVITTSTRTWALPTFNNHLYK |
| | | | QISSQSGASNDNHFFGYSTPWGYFDFNRFHCHFSPRDWORLIN |
| | | | NNWGFRPKRLNFKLFNIQVKEVTQNDGTTTIANNLTSTVQVFT |
| | | | DSEYQLPYVLGSAHQGCLPPFPADVFMVPQYGYLTLNNGSQAV |
| | | | GRSSFYCLEYFPSQMLRTGNNFTFSYTFEDVPFHSSYAHSQSL |
| | | | DRLMNPLIDQYLYFLSRTNTPSGTTTOSRLOFSQAGASDIRDO |
| | | | SRNWLPGPCYRQQRVSKTSADNNNSEFSWTGATKYHLNGRDSL |
| | | | VNPGPAMASHKDDEEKFFPQSGVLIFGKQGSEKTNVDIEKVMI |
| | | | 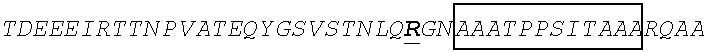 |
| | | | TADVNTQGVLPGMVWQDRDVYLOGPIWAKIPHTDGHFHPSPLM |
| | | | GGFGLKHPPPQILIKNTPVPANPSTTFSAAKFASFITQYSTGQ |
| | | | VSVEIEWELQKENSKRWNPEIQFTSNYNKSVNVDFTVDTNGVY |
| | | | SEPRPIGTRFLTRNL |
| 15 | CGESSYL | 7 | MAADGYLPDWLEDTLSEGIRQWWKLKPGPPPPKPAERHKDDSR |
| | | | GLVLPGYKYLGPFNGLDKGEPVNEADAAALEHDKAYDROLDSG |
| | | | DNPYLKYNHADAEFQERLKEDTSFGGNLGRAVFQAKKRVLEPL |
| | | | GLVEEPVKTAPGKKRPVEHSPVEPDSSSGTGKAGQQPARKRLN |
| | | | FGQTGDADSVPDPQPLGQPPAAPSGLGTNTMATGSGAPMADNN |
| | | | EGADGVGNSSGNWHCDSTWMGDRVITTSTRTWALPTFNNHLYK |
| | | | QISSQSGASNDNHFFGYSTPWGYFDFNRFHCHFSPRDWORLIN |
| | | | NNWGFRPKRLNFKLFNIQVKEVTONDGTTTIANNLTSTVQVFT |
| | | | DSEYQLPYVLGSAHQGCLPPFPADVFMVPQYGYLTLNNGSQAV |
| | | | GRSSFYCLEYFPSQMLRTGNNFTFSYTFEDVPFHSSYAHSQSL |
| | | | DRLMNPLIDQYLYFLSRTNTPSGTTTQSRLQFSQAGASDIRDQ |
| | | | SRNWLPGPCYRQQRVSKTSADNNNSEFSWTGATKYHLNGRDSL |
| | | | VNPGPAMASHKDDEEKFFPQSGVLIFGKOGSEKTNVDIEKVMI |
| | | | 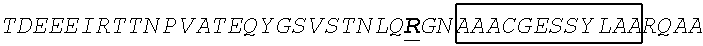 |
| | | | TADVNTQGVLPGMVWQDRDVYLOGPIWAKIPHTDGHFHPSPLM |
| | | | GGFGLKHPPPQILIKNTPVPANPSTTFSAAKFASFITQYSTGO |
| | | | VSVEIEWELQKENSKRWNPEIQFTSNYNKSVNVDFTVDTNGVY |
| | | | SEPRPIGTRFLTRNL |
| 16 | PRTPHTA | 8 | MAADGYLPDWLEDTLSEGIRQWWKLKPGPPPPKPAERHKDDSR |
| | | | GLVLPGYKYLGPFNGLDKGEPVNEADAAALEHDKAYDRQLDSG |
| | | | DNPYLKYNHADAEFQERLKEDTSFGGNLGRAVFQAKKRVLEPL |
| | | | GLVEEPVKTAPGKKRPVEHSPVEPDSSSGTGKAGQQPARKRLN |
| | | | FGQTGDADSVPDPOPLGQPPAAPSGLGTNTMATGSGAPMADNN |
| | | | EGADGVGNSSGNWHCDSTWMGDRVITTSTRTWALPTFNNHLYK |
| | | | QISSQSGASNDNHFFGYSTPWGYFDFNRFHCHFSPRDWORLIN |
| | | | NNWGFRPKRLNFKLFNIQVKEVTONDGTTTIANNLTSTVQVFT |
| | | | DSEYQLPYVLGSAHQGCLPPFPADVFMVPQYGYLTLNNGSQAV |
| | | | GRSSFYCLEYFPSQMLRTGNNFTFSYTFEDVPFHSSYAHSQSL |
| | | | DRLMNPLIDQYLYFLSRTNTPSGTTTQSRLQFSQAGASDIRDO |
| | | | SRNWLPGPCYRQQRVSKTSADNNNSEFSWTGATKYHLNGRDSL |
| | | | VNPGPAMASHKDDEEKFFPQSGVLIFGKQGSEKTNVDIEKVMI |
| | | | 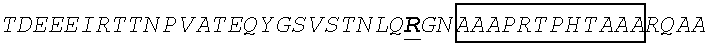 |
| | | | TADVNTQGVLPGMVWODRDVYLQGPIWAKIPHTDGHFHPSPLM |
| | | | GGFGLKHPPPQILIKNTPVPANPSTTFSAAKFASFITQYSTGQ |
| | | | VSVEIEWELQKENSKRWNPEIQFTSNYNKSVNVDFTVDINGVY |
| | | | SEPRPIGTRFLTRNL |
| 17 | ELCDGFA | 9 | MAADGYLPDWLEDTLSEGIRQWWKLKPGPPPPKPAERHKDDSR |
| | | | GLVLPGYKYLGPFNGLDKGEPVNEADAAALEHDKAYDRQLDSG |
| | | | DNPYLKYNHADAEFQERLKEDTSFGGNLGRAVFQAKKRVLEPL |
| | | | GLVEEPVKTAPGKKRPVEHSPVEPDSSSGTGKAGQQPARKRLN |
| | | | FGQTGDADSVPDPOPLGQPPAAPSGLGTNTMATGSGAPMADNN |
| | | | EGADGVGNSSGNWHCDSTWMGDRVITTSTRTWALPTFNNHLYK |
| | | | QISSQSGASNDNHFFGYSTPWGYFDENRFHCHFSPRDWORLIN |
| | | | NNWGFRPKRLNFKLFNIQVKEVTQNDGTTTIANNLTSTVQVFT |
| | | | DSEYQLPYVLGSAHQGCLPPFPADVFMVPQYGYLTLNNGSQAV |
| | | | GRSSFYCLEYFPSQMLRTGNNFTFSYTFEDVPFHSSYAHSQSL |
| | | | DRLMNPLIDQYLYFLSRTNTPSGTTTQSRLQFSQAGASDIRDQ |
| | | | SRNWLPGPCYRQQRVSKTSADNNNSEFSWTGATKYHLNGRDSL |
| | | | VNPGPAMASHKDDEEKFFPQSGVLIFGKQGSEKTNVDIEKVMI |
| | | | 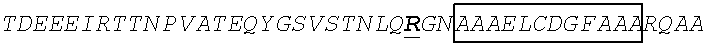 |
| | | | TADVNTQGVLPGMVWODRDVYLQGPIWAKIPHTDGHFHPSPLM |
| | | | GGFGLKHPPPQILIKNTPVPANPSTTFSAAKFASFITQYSTGO |
| | | | VSVEIEWELQKENSKRWNPEIQFTSNYNKSVNVDFTVDINGVY |
| | | | SEPRPIGTRFLTRNL |

TABLE 3
Homologous positions of potential oligomer insertion sites and Y-F and T-V point mutation
sites, respectively, on the capsid proteins of VP3 in different AAV serotypes.

| | | | | | | | | AAV | AAV |
|---|---|---|---|---|---|---|---|---|---|
| Position | AAV1 | AAV2 | AAV5 | AAV6 | AAV7 | AAV8 | AAV9 | Rh10 | Anc80 |
| #1 | G454 | G453 | G446 | G454 | G456 | G456 | G455 | G456 | G454 |
| #2 | P591 | R588 | S576 | P591 | T590 | T591 | A589 | A591 | T589 |
| #3 | Y252 | Y252 | Y242 | Y252 | Y253 | Y253 | Y252 | Y253 | Y252 |
| #4 | Y273 | Y272 | Y263 | Y273 | Y274 | Y275 | Y274 | Y275 | Y273 |
| #5 | Y445 | Y444 | R437 | Y445 | Y446 | Y447 | Y446 | Y447 | Y445 |
| #6 | F501 | Y500 | S487 | F501 | F503 | F503 | F501 | F503 | F501 |
| #7 | Y701 | Y700 | Y689 | Y701 | Y702 | Y703 | Y701 | Y703 | Y701 |
| #8 | Y705 | Y704 | Y693 | Y705 | F706 | Y707 | Y705 | Y707 | Y705 |
| #9 | Y731 | Y730 | Y719 | Y731 | Y732 | Y733 | Y731 | Y733 | Y731 |
| #10 | T492 | T491 | G478 | T492 | T494 | T494 | T492 | T494 | T492 |

Underlined positions exhibit different amino acids with respect to AAV2.

TABLE 4
Capsid gene sequence (VP1) of adeno-as-
sociated virus 2 (Y252, 272, 444, 500,
700, 730F), referred to as AAV2(M6)

| SEQ | |
|---|---|
| ID | |
| NO | Sequence |
| 27 | ATGGCTGCCGATGGTTATCTTCCAGATTGGCTCGAGGACACTCTCT |
| | CTGAAGGAATAAGACAGTGGTGGAAGCTCAAACCTGGCCCACCACC |
| | ACCAAAGCCCGCAGAGCGGCATAAGGACGACAGCAGGGGTCTTGTG |
| | CTTCCTGGGTACAAGTACCTCGGACCCTTCAACGGACTCGACAAGG |
| | GAGAGCCGGTCAACGAGGCAGACGCCGCGGCCCTCGAGCACGACAA |
| | AGCCTACGACCGGCAGCTCGACAGCGGAGACAACCCGTACCTCAAG |
| | TACAACCACGCCGACGCGGAGTTTCAGGAGCGCCTTAAAGAAGATA |
| | CGTCTTTTGGGGGCAACCTCGGACGAGCAGTCTTCCAGGCGAAAAA |
| | GAGGGTTCTTGAACCTCTGGGCCTGGTTGAGGAACCTGTTAAGACG |
| | GCTCCGGGAAAAAAGAGGCCGGTAGAGCACTCTCCTGTGGAGCCAG |
| | ACTCCTCCTCGGGAACCGGAAAGGCGGGCCAGCAGCCTGCAAGAAA |
| | AAGATTGAATTTTGGTCAGACTGGAGACGCAGACTCAGTACCTGAC |
| | CCCCAGCCTCTCGGACAGCCACCAGCAGCCCCCTCTGGTCTGGGAA |
| | CTAATACG |
| | ATGGCTACAGGCAGTGGCGCACCAATGGCAG |
| | ACAATAACGAGGGCGCCGACGGAGTGGGTAA |
| | TTCCTCCGGAAATTGGCATTGCGATTCCACA |
| | TGGATGGGCGACAGAGTCATCACCACCAGCA |
| | CCCGAACCTGGGCCCTGCCCACCTTCAACAA |
| | CCACCTGTACAAACAAATTTCCAGCCAATCA |
| | GGAGCTTCGAACGACAATCACTTCTTTGGCT |
| | ACAGCACCCCTTGGGGGTATTTTGACTTCAA |
| | CAGATTCCACTGCCACTTTTCACCACGTGAC |
| | TGGCAAAGACTCATCAACAACAACTGGGGAT |
| | TCCGACCCAAGAGACTCAACTTCAAGCTCTT |
| | TAACATTCAAGTCAAAGAGGTCACGCAGAAT |
| | GACGGTACGACGACGATTGCCAATAACCTTA |
| | CCAGCACGGTTCAGGTGTTTACTGACTCGGA |
| | GTACCAGCTCCCGTACGTCCTCGGCTCGGCG |
| | CATCAAGGATGCCTCCCGCCGTTCCCAGCAG |
| | ACGTCTTCATGGTGCCACAGTATGGATACCT |
| | CACCCTGAACAACGGGAGTCAGGCAGTAGGA |
| | CGCTCTTCATTTTACTGCCTGGAGTACTTTC |
| | CTTCTCAGATGCTGCGTACCGGAAACAACTT |
| | TACCTTCAGCTACACTTTTGAGGACGTTCCT |
| | TTCCACAGCAGCTACGCTCACAGCCAGAGTC |
| | TGGACCGTCTCATGAATCCTCTCATCGACCA |
| | GTACCTGTATTTCTTAAGCAGAACAAACACT |
| | CCAAGTGGAACCACCACGCAGTCAAGGCTTC |
| | AGTTTTCTCAGGCCGGAGCGAGTGACATTCG |
| | GGACCAGTCTAGGAACTGGCTTCCTGGACCC |
| | TGTTACCGCCAGCAGCGAGTATCAAAGACAT |
| | CTGCGGATAACAACAACAGTGAATTCTCGTG |
| | GACCGGTGCTACCAAGTACCACCTCAATGGC |
| | AGAGACTCTCTGGTGAATCCGGGCCCGGCCA |
| | TGGCAAGCCACAAGGACGATGAAGAAAAGTT |
| | TTTTCCTCAGAGCGGGGTTCTCATCTTTGGG |
| | AAGCAAGGCTCAGAGAAAACAAATGTGGACA |
| | TTGAAAAGGTCATGATTACAGACGAAGAGGA |
| | AATCAGGACAACCAATCCCGTGGCTACGGAG |
| | CAGTATGGTTCTGTATCTACCAACCTCCAGA |
| | GAGGCAACAGACAAGCAGCTACCGCAGATGT |
| | CAACACACAAGGCGTTCTTCCAGGCATGGTC |
| | TGGCAGGACAGAGATGTGTACCTTCAGGGGC |
| | CCATCTGGGCAAAGATTCCACACACGGACGG |
| | ACATTTTCACCCCTCTCCCCTCATGGGTGGA |
| | TTCGGACTTAAACACCCTCCTCCACAGATTC |
| | TCATCAAGAACACCCCGGTACCTGCGAATCC |
| | TTCGACCACCTTCAGTGCGGCAAAGTTTGCT |
| | TCCTTCATCACACAGTACTCCACGGGACAGG |
| | TCAGCGTGGAGATCGAGTGGGAGCTGCAGAA |
| | GGAAAACAGCAAACGCTGGAATCCCGAAATT |
| | CAGTTCACTTCGAACTACAACAAGTCTGTTA |
| | ATGTGGACTTTACTGTGGACACTAATGGCGT |
| | GTATTCAGAGCCTCGCCCCATTGGTACCAGA |
| | TTCCTGACTCGTAATCTGTAA |

Based on the AAV 2 complete genome sequence, GenBank accession number J01901.1

The Cap gene of the library consists of VP3 (bold sequence), tyrosine to phenylalanine (Y-F) mutations are underlined, base pair numbering refers to the whole VP1 sequence.

1-17. (canceled)

18. An adeno-associated virus (AAV) capsid polypeptide comprising a peptide insert at position 453 or at position 587 to 592 of the AAV serotype 2 capsid or a position homologous thereto in an AAV of another serotype, wherein the peptide insert is selected from:
(Cap3; SEQ ID NO: 10)
SASEAST,
(Cap5; SEQ ID NO: 11)
DTRPHDQ,
(Cap7; SEQ ID NO: 12)
EHYNSTC,
(Cap9; SEQ ID NO: 13)
PNPNCTL,
(Cap11; SEQ ID NO: 14)
TPPSITA,
(Cap12; SEQ ID NO: 15)
CGESSYL,
(Cap13; SEQ ID NO: 16)
PRTPHTA
and
(Cap14; SEQ ID NO: 17)
ELCDGFA.

19. The method of claim 18, wherein the insert is at position 587 to 592 of the AAV serotype 2 capsid or a position homologous thereto in an AAV of another serotype.

20. The method of claim 19, wherein the insert is at position 587 of the AAV serotype 2 capsid or a position homologous thereto in an AAV of another serotype.

21. The method of claim 18, wherein the insert is selected from SASEAST (Cap3; SEQ ID NO: 10), TPPSITA (Cap11; SEQ ID NO: 14), PRTPHTA (Cap13; SEQ ID NO: 16) and ELCDGFA (Cap14; SEQ ID NO: 17).

22. An adeno-associated virus capsid polypeptide, comprising a sequence having at least (≥) 85% identity to SEQ ID NO: 001, wherein the polypeptide comprises:
an insert at position 587, 588, 589, 590, 591 or 592, and
the insert comprises a peptide sequence selected from any one of SEQ ID NOs: 10, 11, 12, 13, 14, 15, 16, and 17.

23. The AAV capsid polypeptide of claim 22, comprising a sequence having 90% identity to SEQ ID NO: 001.

24. The AAV capsid polypeptide of claim 22, comprising a sequence having 95% identity to SEQ ID NO: 001.

25. The AAV capsid polypeptide of claim 22 wherein the polypeptide comprises an insert at position 587, 588 or 589.

26. The AAV capsid polypeptide of claim 25, wherein the polypeptide comprises an insert at position 587.

27. The AAV capsid polypeptide of claim 22, wherein the AAV capsid polypeptide consists of a sequence having 85% identity to SEQ ID NO: 001.

28. The AAV capsid polypeptide of claim 22, wherein the AAV capsid polypeptide consists of a sequence having 90% identity to SEQ ID NO: 001.

29. The AAV capsid polypeptide of claim 22, wherein the AAV capsid polypeptide consists of a sequence having 95% identity to SEQ ID NO: 001.

30. The adeno-associated virus capsid polypeptide according to claim 22, wherein the polypeptide has at least 90% of the biological activity of a sequence selected from SEQ ID NO: 2-SEQ ID NO: 9.

31. The adeno-associated virus capsid polypeptide according to claim 22, wherein the polypeptide comprises a sequence selected from SEQ ID NO: 2-SEQ ID NO: 9.

32. The adeno-associated virus capsid polypeptide according to claim 22, wherein the adeno-associated virus capsid polypeptide consists of an amino acid sequence selected from SEQ ID NO: 2-SEQ ID NO: 9.

33. The adeno-associated virus capsid polypeptide of claim 18 or 22, wherein the AAV capsid protein is an AAV2 capsid comprising:
a. one or more tyrosine to phenylalanine substitutions at positions 252, 272, 444, 500, 700, 704 and 730, and/or
b. one or more threonine to valine substitutions, or
wherein the capsid is an AAV capsid other than an AAV2 capsid, comprising one or more tyrosine to phenylalanine substitutions and/or one or more threonine to valine substitutions at homologous positions in the AAV2 capsid and the AAV capsid.

34. The AAV capsid polypeptide of claim 33, wherein the one or more tyrosine to phenylalanine substitutions are at positions 252, 272, 444, 500, 704 and 730.

35. The AAV capsid polypeptide of claim 33, wherein the one or more tyrosine to phenylalanine substitutions are at all of the positions 252, 272, 444, 500, 700 and 730.

36. The AAV capsid polypeptide of claim 33, wherein the one or more threonine to valine substitutions is T491V.

37. A nucleic acid sequence encoding the AAV capsid polypeptide according to claim 18 or 22.

38. The nucleic acid sequence of claim 37, wherein the sequence is a self-complementary or single stranded vector genome.

39. The nucleic acid sequence of claim 38, wherein the sequence is a self-complementary vector genome.

40. The nucleic acid sequence of claim 37 or claim 38, wherein the nucleic acid sequence comprises a transgene.

41. The nucleic acid sequence of claim 40, wherein the transgene encodes the sequence of a protein, a siRNA, a shRNA or a CRISPR/Cas-gRNA cassette.

42. The nucleic acid sequence of claim 41, wherein the transgene encodes a light-sensitive protein.

43. The nucleic acid sequence of claim 41, wherein the transgene is under control of a promoter sequence operable in a mammalian cell.

44. The nucleic acid sequence of claim 43, wherein the mammalian cell is a retinal cell.

45. The nucleic acid sequence of claim 44, wherein the retinal cell is a human retinal cell.

46. The nucleic acid sequence of claim 43, wherein the promoter is a ubiquitous or cell-specific promoter.

47. The nucleic acid sequence of claim 43, wherein the promoter is selected from a CMV immediate early promoter, or hEfla promoter.

48. A method of treating a condition in a subject in need thereof, comprising administering the AAV capsid polypeptide of claim 18 or claim 22.

49. The method of claim 48, wherein the condition affects a retinal or retinal pigment epithelium cell, a photoreceptor, a bipolar cell, a ganglion cell, and/or an amacrine cell.

50. The method of claim 48, wherein the AAV capsid polypeptide is administered by intravitreal administration or by subretinal administration.

51. The method of claim 48, wherein the intravitreal administration is intravitreal injection.


SEQUENCE LISTING (28 sequences)

SEQ ID NO: 1 - protein, 735 aa, Artificial Sequence
  AAV2 capsid protein VP1 with different mutations
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTFNNHLYKQISSQSGASNDNHFFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQRVSKTSADNNNSEFSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQFTSNYNKSVNVDFTVDTNGVYSEPRPIGTRFLTRNL

SEQ ID NO: 2 - protein, 747 aa, Artificial Sequence
  AAV2 capsid protein VP1 with insertion
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTFNNHLYKQISSQSGASNDNHFFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQRVSKTSADNNNSEFSWTGATKYHLNGRDSLVNPGPAMASHKDDEEKFFPQSGVLIFGKQGSEKTNVDIEKVMITDEEEIRTTNPVATEQYGSVSTNLQRGNAAASASEASTAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQFTSNYNKSVNVDFTVDTNGVYSEPRPIGTRFLTRNL

SEQ ID NO: 3 - protein, 747 aa, Artificial Sequence
  AAV2 capsid protein VP1 with insertion
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTFNNHLYKQISSQSGASNDNHFFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQRVSKTSADNNNSEFSWTGATKYHLNGRDSLVNPGPAMASHKDDEEKFFPQSGVLIFGKQGSEKTNVDIEKVMITDEEEIRTTNPVATEQYGSVSTNLQRGNAAADTRPHDQAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQFTSNYNKSVNVDFTVDTNGVYSEPRPIGTRFLTRNL

SEQ ID NO: 4 - protein, 747 aa, Artificial Sequence
  AAV2 capsid protein VP1 with insertion
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTFNNHLYKQISSQSGASNDNHFFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQRVSKTSADNNNSEFSWTGATKYHLNGRDSLVNPGPAMASHKDDEEKFFPQSGVLIFGKQGSEKTNVDIEKVMITDEEEIRTTNPVATEQYGSVSTNLQRGNAAAEHYNSTCAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQFTSNYNKSVNVDFTVDTNGVYSEPRPIGTRFLTRNL

SEQ ID NO: 5 - protein, 747 aa, Artificial Sequence
  AAV2 capsid protein VP1 with insertion
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTFNNHLYKQISSQSGASNDNHFFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQRVSKTSADNNNSEFSWTGATKYHLNGRDSLVNPGPAMASHKDDEEKFFPQSGVLIFGKQGSEKTNVDIEKVMITDEEEIRTTNPVATEQYGSVSTNLQRGNAAAPNPNCTLAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQFTSNYNKSVNVDFTVDTNGVYSEPRPIGTRFLTRNL

SEQ ID NO: 6 - protein, 747 aa, Artificial Sequence
  AAV2 capsid protein VP1 with insertion
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTFNNHLYKQISSQSGASNDNHFFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQRVSKTSADNNNSEFSWTGATKYHLNGRDSLVNPGPAMASHKDDEEKFFPQSGVLIFGKQGSEKTNVDIEKVMITDEEEIRTTNPVATEQYGSVSTNLQRGNAAATPPSITAAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQFTSNYNKSVNVDFTVDTNGVYSEPRPIGTRFLTRNL

SEQ ID NO: 7 - protein, 747 aa, Artificial Sequence
  AAV2 capsid protein VP1 with insertion
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTFNNHLYKQISSQSGASNDNHFFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQRVSKTSADNNNSEFSWTGATKYHLNGRDSLVNPGPAMASHKDDEEKFFPQSGVLIFGKQGSEKTNVDIEKVMITDEEEIRTTNPVATEQYGSVSTNLQRGNAAACGESSYLAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQFTSNYNKSVNVDFTVDTNGVYSEPRPIGTRFLTRNL

SEQ ID NO: 8 - protein, 747 aa, Artificial Sequence
  AAV2 capsid protein VP1 with insertion
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTFNNHLYKQISSQSGASNDNHFFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQRVSKTSADNNNSEFSWTGATKYHLNGRDSLVNPGPAMASHKDDEEKFFPQSGVLIFGKQGSEKTNVDIEKVMITDEEEIRTTNPVATEQYGSVSTNLQRGNAAAPRTPHTAAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQFTSNYNKSVNVDFTVDTNGVYSEPRPIGTRFLTRNL

SEQ ID NO: 9 - protein, 747 aa, Artificial Sequence
  AAV2 capsid protein VP1 with insertion
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTFNNHLYKQISSQSGASNDNHFFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQRVSKTSADNNNSEFSWTGATKYHLNGRDSLVNPGPAMASHKDDEEKFFPQSGVLIFGKQGSEKTNVDIEKVMITDEEEIRTTNPVATEQYGSVSTNLQRGNAAAELCDGFAAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQFTSNYNKSVNVDFTVDTNGVYSEPRPIGTRFLTRNL

SEQ ID NO: 10 - protein, 7 aa, Artificial Sequence
  heptamer insert
SASEAST

SEQ ID NO: 11 - protein, 7 aa, Artificial Sequence
  heptamer insert
DTRPHDQ

SEQ ID NO: 12 - protein, 7 aa, Artificial Sequence
  heptamer insert
EHYNSTC

SEQ ID NO: 13 - protein, 7 aa, Artificial Sequence
  heptamer insert
PNPNCTL

SEQ ID NO: 14 - protein, 7 aa, Artificial Sequence
  heptamer insert
TPPSITA

SEQ ID NO: 15 - protein, 7 aa, Artificial Sequence
  heptamer insert
CGESSYL

SEQ ID NO: 16 - protein, 7 aa, Artificial Sequence
  heptamer insert
PRTPHTA

SEQ ID NO: 17 - protein, 7 aa, Artificial Sequence
  heptamer insert
ELCDGFA

SEQ ID NO: 18 - protein, 7 aa, Artificial Sequence
  heptamer insert
HHNDRAP

SEQ ID NO: 19 - protein, 7 aa, Artificial Sequence
  heptamer insert
DGNLHKS

SEQ ID NO: 20 - protein, 7 aa, Artificial Sequence
  heptamer insert
HSDSSKP

SEQ ID NO: 21 - protein, 7 aa, Artificial Sequence
  heptamer insert
TLTGLCP

SEQ ID NO: 22 - protein, 7 aa, Artificial Sequence
  heptamer insert
NHAPNHC

SEQ ID NO: 23 - protein, 7 aa, Artificial Sequence
  heptamer insert
DRRPGIC

SEQ ID NO: 24 - protein, 7 aa, Artificial Sequence
  heptamer insert
VDATCMQ

SEQ ID NO: 25 - DNA, 54 bp, Artificial Sequence
  single-stranded randomized 7mer pool with NNB codons
  misc_feature (14)..(15): n is a, c, g, or t
  misc_feature (17)..(18): n is a, c, g, or t
  misc_feature (20)..(21): n is a, c, g, or t
  misc_feature (23)..(24): n is a, c, g, or t
  misc_feature (26)..(27): n is a, c, g, or t
  misc_feature (29)..(30): n is a, c, g, or t
  misc_feature (32)..(33): n is a, c, g, or t
ttggcgcgccgcvnnvnnvnnvnnvnnvnnvnnggcggccgcttttttccttga

SEQ ID NO: 26 - DNA, 15 bp, Artificial Sequence
  antisense primer
ctcaaggaaaaaagc

SEQ ID NO: 27 - DNA, 2208 bp, adeno-associated virus 2
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctccggaaattggcattgcgattccacatggatgggcgacagagtcatcaccaccagcacccgaacctgggccctgcccaccttcaacaaccacctgtacaaacaaatttccagccaatcaggagcttcgaacgacaatcacttctttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtatttcttaagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaattctcgtggaccggtgctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagttcacttcgaactacaacaagtctgttaatgtggactttactgtggacactaatggcgtgtattcagagcctcgccccattggtaccagattcctgactcgtaatctgtaa

SEQ ID NO: 28 - protein, 12 aa, Artificial Sequence
  alanin spacer
AAASASEASTAA